(19) United States
(12) Patent Application Publication
(10) Pub. No.: US 2019/0274759 A1
(43) Pub. Date: Sep. 12, 2019

(54) DERMATOLOGICAL DIAGNOSIS AND TREATMENT SYSTEM

(71) Applicants: UNIVERSITE DE BORDEAUX, BORDEAU (FR); CENTRE NATIONAL DE LA RECHERCHE SCIENTIFIQUE, PARIS (FR); IRISIOME, TALENCE (FR); INSTITUT D'OPTIQUE GRADUATE SCHOOL, PALAISEAU (FR)

(72) Inventors: Romain ROYON, DAX (FR); Giorgio SANTARELLI, Talence (FR); Jérôme LHERMITE, ANGLET (FR); Romain DUBRASQUET, SABRES (FR)

(21) Appl. No.: 16/319,719

(22) PCT Filed: Jul. 21, 2017
(86) PCT No.: PCT/FR2017/052012

§ 371 Date: Jan. 22, 2019

(30) Foreign Application Priority Data
Jul. 22, 2016 (FR) 1657051

(51) Int. Cl. A61B 18/20; A61B 5/00; A61N 5/06

(52) U.S. Cl. CPC A61B 18/203; A61B 5/0075; A61B 2017/00769; A61B 5/4836; A61N 5/0616; A61B 5/441

(57) ABSTRACT

Disclosed is a dermatological diagnostic and treatment system including a light source device, a light flow collecting device, a spectroscopic measurement device, and a computer. The light source device includes a laser source tunable in wavelength inside the spectral range, the laser source being tunable in duration and/or rate and the laser source being adapted to generate a second treatment laser beam of high intensity at said first wavelength towards the epidermal surface. The dermatological diagnostic and treatment system further includes a temperature sensor and a feedback device, the temperature sensor being arranged to record a temperature measurement signal of the epidermal surface as a function of the application of the second laser beam. The feedback device is configured to modify the duration and/or rate of the laser pulses as a function of the temperature measurement signal.

### TECHNICAL FIELD TO WHICH THE INVENTION RELATES

The present invention generally relates to the field of dermatological diagnostic and/or treatment devices.

It more particularly relates to an optical diagnostic and laser treatment system applied to dermatology.

It relates in particular to a diagnostic and treatment device for the modification of cutaneous chromophores, for example for tattoo removal.

### TECHNOLOGICAL BACK-GROUND

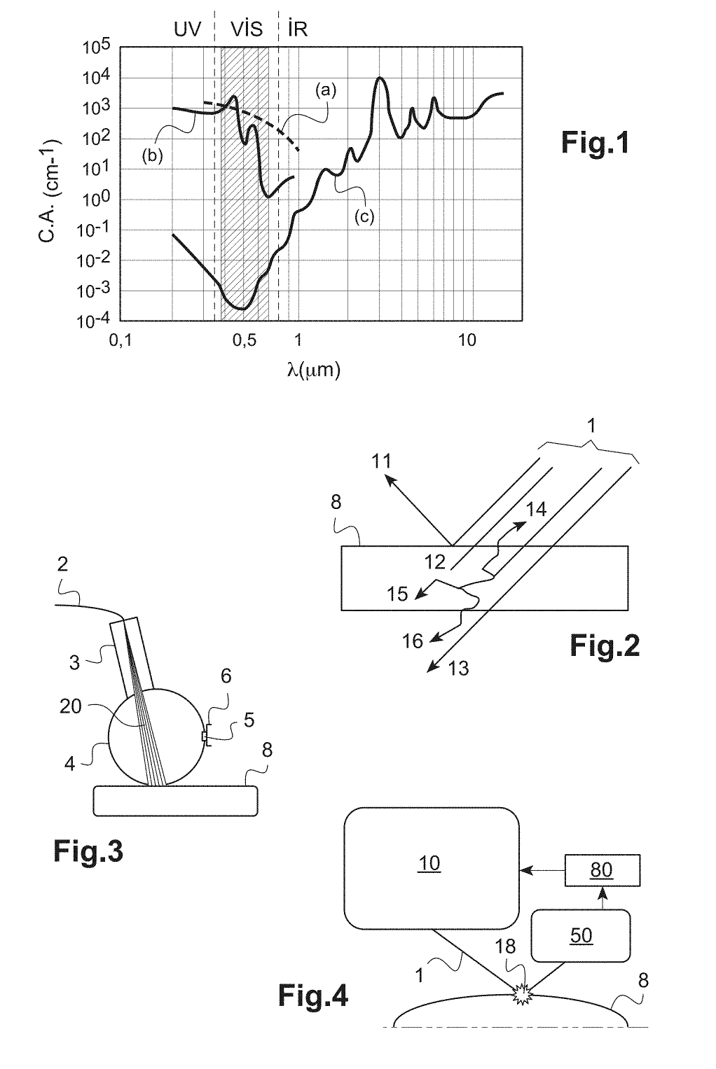

The laser dermatological treatments are generally based on the absorption of a laser wave by different cutaneous chromophores. Some chromophores are endogenous, such as melanin, haemoglobin and water. Other chromophores are exogenous, such as tattoo pigments. When a sufficient energy is absorbed by a chromophore, the latter is naturally destructed and eliminated by physiological processes implementing macrophages. FIG. 1 shows spectroscopic measurements of the absorption coefficient (CA) of the main chromophores of the epidermal tissues, respectively melanin in (a) in FIG. 1, haemoglobin in (b) and water in (c). The absorption coefficient measurements are herein shown as a function of the wavelength (λ) in the spectral domain extending from the ultraviolet (UV, from 100 nm to 350 nm) to the visible (VIS, from 350 nm to 800 nm) and to the near infrared (NIR, from 850 nm to 1.5 μm) or even to the far infrared (IF from 1.5 μm to 10 μm) for water.

The document US 2009/0227994 A1 describes a device for eliminating a compound, for example a tattoo pigment, from a skin tissue. The document US 2001/0056237 A1 describes a device and a method for controlling in vivo and non-invasively the presence of one or several chromophores in a sample of skin tissue, of epithelial or sub-epithelial tissue. The document US 2007/0197883 A1 describes a diagnostic device for measuring a spectrum of diffuse reflection or of Raman diffusion of tattoo pigments.

At present, a conventional dermatological laser treatment device uses pulses whose duration is comprised between a few hundreds of picoseconds and several nanoseconds and of high energies (from 200 mJ to several Joules). The dermatological laser treatments generally use thermal effects that may generate significant postoperative traumas. The thermal effect being not specific, this effect corresponds to a burning by carbonization of the targets without respecting the surrounding tissues. This is due to the fact that the laser wave used does not specifically corresponds to a peak of absorption and that a lot of energy is required to deteriorate the target, causing damages to the immediate environment. Many postoperative traumas appear, such as hyperpigmentation (darker skin), hypopigmentation (clearer skin), skin discoloration, scar and/or infection. The treatment of these secondary effects is performed during the healing, when the skin is reconstituted.

Now, the choice of the wavelength used for a laser treatment is essential in dermatology. Indeed, the absorption and penetration of the light strongly depend on the wavelength (FIG. 1). The complete range of lasers available on the market covers a great number of wavelengths, in particular thanks to the arrival of laser diodes. However, if a practitioner wants to treat a great number of pathologies and a great number of patients with different phototypes and different tattoos, he must have an extended laser park. This represents an investment of several hundreds of thousands euros with a maintenance cost of about 10% of the purchasing price per year. Finally, the fact to have only one laser, as it is often the case, does not allow rubbing out all the inks during a treatment aiming at removing a tattoo. Consequently, the efficiency of a laser treatment to remove a tattoo is poor, in particular on colour tattoos.

It is known in particular from the document US 2007/0197883 a spectroscopic diagnostic device as an aid for laser tattoo removing. This diagnostic device includes an excitation laser and a Raman spectrometer, which performs a Raman absorption measurement on the tattooed skin in order to determine the Raman absorption lines. The diagnostic device further includes a wideband light source and a spectrometer to measure a diffuse reflectance spectrum. The Raman spectrum is used to determine the composition of the pigments of a tattoo. The diffuse reflectance spectrum is used to estimate an absorption rate of a tattoo removing laser at a determined wavelength. The Raman and diffuse reflectance spectrums are used to select the parameters of a detattooing laser before or during the laser treatment. The device allows selecting a Q-switch laser having a wavelength, a pulse energy, a peak power and a rate that are adapted to the tattoo removal laser treatment of the skin analysed. In the present document, the rate is equivalent to the repetition frequency of the laser pulses.

It is also known from the document WO 98/24514 a dermatological treatment method and device comprising a laser controlled by a temperature sensor used for detecting epidermal injuries induced by the laser treatment. According to this document, a control loop acts on the energy, the pulse duration and/or the rate of the laser so as to avoid injuring the epidermis.

At present, there exists no dermatological laser treatment device able to automatically determine and adapt in real time the optimum treatment conditions.

There exists a need for a diagnostic and laser treatment device making it possible to locally remove a tattoo pigment on a particular epidermis without risk of injury of the surrounding epidermal tissues.

There exists a need for a single diagnostic and laser treatment device making it possible to remove different tattoo pigments on different types of epidermis without risk of injury of the surrounding epidermal tissues.

### OBJECT OF THE INVENTION

In order to remedy the above-mentioned drawback of the state of the art, the present invention proposes a dermatological diagnostic and treatment system comprising a light source device, a light flow collecting device, an absorption spectroscopic measurement device and a calculator.

More particularly, it is proposed according to the invention a dermatological diagnostic and treatment system wherein the light source device is adapted to generate a first light beam of low intensity (from 1 mW to 100 mW of mean power) towards an epidermal surface of a patient, the first light beam extending over a spectral range or being tunable in wavelength over a spectral range, the light flow collecting device being arranged to collect a first light flow by reflection and/or scattering of the first light beam on said epidermal surface, the absorption spectroscopic measurement device being adapted to record a first spectroscopic measurement of the first light flow as a function of the spectral range, the calculator being adapted to receive the first spectroscopic measurement in the spectral range and to determine a first wavelength associated with an absorption of a pigment of said epidermal surface and a second wavelength associated with an absorption of another tissue constituent of said epidermal surface, and wherein the light source device includes a laser source tunable in wavelength inside the spectral range, the laser source being adapted to generate a second treatment laser beam of high intensity (from 100 mW to 100 W of mean power) at said first wavelength towards said epidermal surface.

This non-invasive system allows treating specifically a cutaneous pigment so as to decompose it by spectrally specific absorption, while strongly limiting the heating of the surrounding epidermal tissue.

Other non-limitative and advantageous features of the dermatologic diagnostic and treatment system according to the invention, taken individually or according to all the technically possible combinations, are the following:
the second laser beam is consisted of laser pulses of duration comprised between 500 femtoseconds (fs) and 100 microseconds (μs) and, preferably, comprised between 30 picoseconds (ps) and 10 nanoseconds (ns);
the second laser beam is consisted of pulses at a rate comprised between 1 hertz (Hz) and 1 gigahertz (GHz) and, preferably, comprised between 10 megahertz (MHz) and 500 MHz;
the second laser beam has a power comprised between 100 milliwatts (mW) and 100 W;
the light source device includes a laser source continuously tunable in wavelength over a spectral range extending over at least 100 nm, and preferably from the ultraviolet to the near infrared, the laser source being adapted to generate the second laser beam;
the light flow collecting device includes a integrating sphere, and the absorption spectroscopic measurement device includes a spectrometer or a monochromator and a photodetector;
the light flow collecting device is configured to collect a second light flow by reflection and/or scattering of the second light beam on said epidermal surface and the absorption spectroscopic measurement device is adapted to record a second light flow measurement signal at the first wavelength;
the dermatological diagnostic and treatment system further includes a temperature sensor arranged to record a temperature measurement signal of said epidermal surface as a function of the application of the second laser beam;
the laser source being further tunable in duration and/or rate, the dermatological diagnostic and treatment system further includes a feedback device configured to modify the duration and/or the rate of the laser pulses as a function of the second light flow measurement signal and/or as a function of the temperature measurement signal;
the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

According to an embodiment, the light source device includes another light source adapted to generate the first light beam. The other light source is preferably chosen among a white-light lamp, a continuous or pulsed light-emitting diode.

According to another embodiment, the light source device includes a single laser source tunable in wavelength, the laser source being adapted to generate the first light beam and the second laser beam and the light source device includes a device for intensity modulating the laser beam generated by the laser source.

The invention also proposes a dermatological diagnostic and treatment method comprising the following steps:
generating a first, low-intensity light beam towards an epidermal surface of a patient,
collecting a first light flow by reflection and/or scattering of the first light beam on said epidermal surface,
spectroscopically analysing the first light flow to deduce therefrom a recording of a first spectroscopic measurement,
determining, by digital processing of the first spectroscopic measurement, a first wavelength associated with an absorption of a pigment of said epidermal surface and a second wavelength associated with another absorption of a tissue of said epidermal surface, and
adjusting a laser pulse source tunable in wavelength to the first wavelength and generating a second, high-intensity treatment laser beam towards said epidermal surface at said first wavelength.

The following description with respect to the appended drawings, given by way of non-limitative examples, will permit a good understanding of what the invention consists in and of how it can be implemented.

In the appended drawings:

FIG. 1 shows spectroscopic measurements of the absorption coefficient (CA) of the main chromophores of the epidermal tissues;

FIG. 2 schematically shows the different physical phenomena of interaction between a laser beam and an epidermal tissue;

FIG. 3 schematically shows a cross-sectional view of a reflection or backscattering spectroscopy measurement device;

FIG. 4 schematically shows a diagnostic and treatment system according to one embodiment;

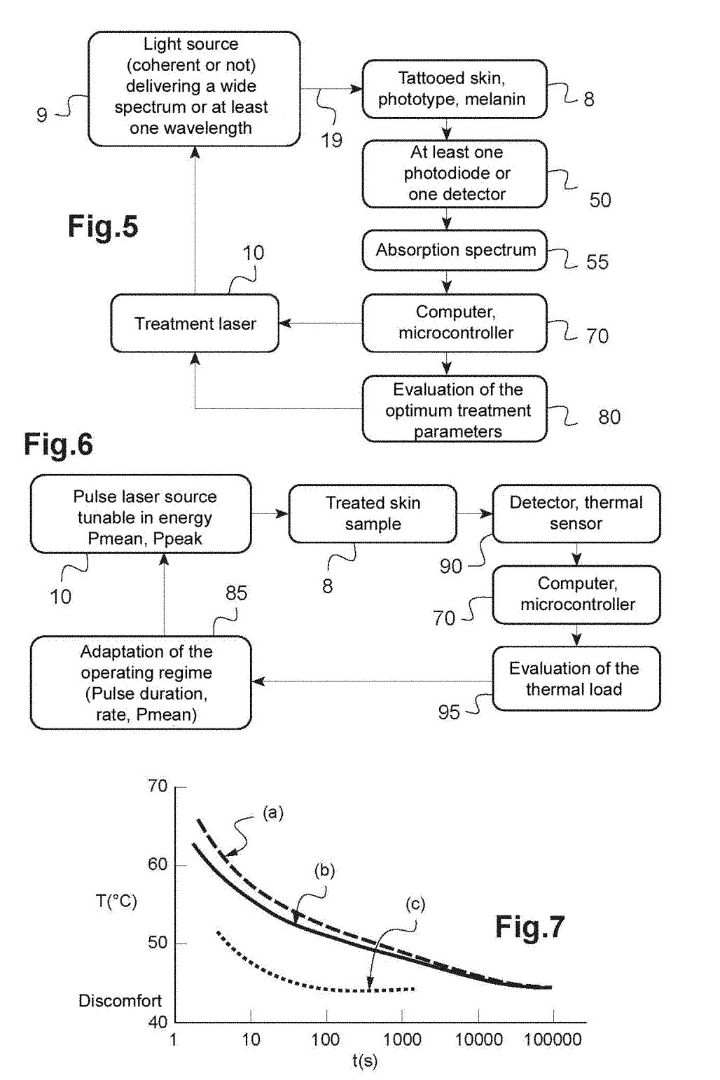

FIG. 5 schematically shows a treatment method according to one embodiment;

FIG. 6 schematically shows a variant of the treatment method according to one embodiment;

FIG. 7 shows a burning severity diagram as a function of the duration of contact and of the temperature.

### DETAILED DESCRIPTION OF AN EXEMPLARY EMBODIMENT

Device

An aspect of the present disclosure consists in determining in what proportion the skin reflects a light radiation so as to determine accurately the laser treatment wavelength the most adapted to the pigment to be decomposed and to the skin of the patient.

Generally, when an incident light beam 1 reaches the surface of an epidermal tissue 8, a portion of the incident beam forms a reflected beam 11. The remaining of the incident beam propagates in the epidermal tissue 8. Another portion 12 is absorbed by the tissue constituents. Still another portion of the incident beam is scattered towards the rear (backscattered beam 14), towards the front (forward-scattered beam 16) and into the epidermal tissue (lateral scattering beam 15). Finally, a last incident beam portion may manage to path through the tissue volume and to form a transmitted beam 13.

The optical reflection at the surface of the tissue limits the efficiency of the laser treatment. As a function of the wavelength and the nature of the surface of the tissues 8, the optical reflection may be specular or diffuse. The surface reflection may reach high values. The reflection coefficient of a laser beam on the skin at a wavelength of 488 nm is evaluated at about 30%. The scattering is an interaction of the light with the material during which the direction of the incident beam is modified. The backscattered beam 14 goes out of the tissue 8. On the other hand, the absorbed beam 12 and the scattered beams 15, 16 transform the energy brought by the incident photons into heat.

The proportion of the reflection, absorption, transmission and scattering phenomena depends, for a given tissue, on the wavelength of the incident beam. In the short ultraviolet and in the far infrared, the absorption is more important than the scattering. In other portions of the spectrum (from 300 nm to 2 μm), the scattering influences the geometry of the beam by producing a lateral widening and by reducing the penetration depth of the laser beam.

In FIG. 3 is shown an absorption spectroscopic measurement device. This device includes a light source device 2, an optical system 3, a device 4 for collecting the reflected and backscattered light beam, a spectrometry device 6 and a photo-detection device 5.

The light source device 2 emits a low-intensity light beam 20 (from 1 mW to 100 mW of mean power). A light source device 2 emitting over a wide spectrum, a spectral band covering a spectral range able to go up to 100 nm, may be used. By way of example, the source device includes a supercontinuum laser. However, it is generally sufficient to perform measurements at a few discrete wavelengths in the spectral range. In another example, the source device includes several laser diodes of low mean power (from 1 mW to 100 mW) adapted to choose different wavelengths in several spectral bands corresponding to different therapeutic windows (visible: 500-700 nm, infrared: about 1 micrometre). The optical system 3 serves to shape the incident light beam and to adapt the size thereof as a function of the desired fluence.

This light source device 2 is operable to illuminate the epidermal tissue 8 to be treated under a low light beam so as to avoid deteriorating the epidermal tissue, and to change the optical reflection, absorption, scattering and transmission properties of the epidermal tissue.

The collecting device 4 allows collecting the light flow reflected or backscattered by the skin. The collecting device 4 is configured so as to collect a maximum of photons under illumination by a low-intensity light source 2. By way of example, the collecting device 4 comprises an integrating sphere.

The spectrometry device 6 includes for example a spectrometer equipped with a detector 5 configured to operate in the spectral domain of the light source 2 as a function of the wavelengths of interest. In another example, the spectrometry device 6 comprises a monochromator equipped with a detector 5, which analyses directly a wavelength reflected by the patient's skin.

The spectrometry 6 and photo-detection 5 device may be integrated to the collecting device 4. As an alternative, the spectrometry 6 and photo-detection 5 device is connected by an optical fibre to the collecting device 4, so as to outset the case of the spectrometry 6 and photo-detection 5 device.

The absorption spectroscopic measurement device is calibrated during the fabrication. A calibration is preferably performed before each later use. The calibration source may be the laser itself, set at a given wavelength or an additional source added in the machine (LED or laser diode). In a first case, the calibration consists in arranging a mirror in front of an opening of the integrating sphere to measure the maximum light intensity reflected. In another example, the calibration consists in arranging a light shaft. The reflection measurements on an epidermal tissue are then added to this maximum light intensity, to obtain relative spectroscopy measurements. The absorption is then calculated by subtracting the reference reflection measurement (with the mirror) from the reflection measurement on the epidermal tissue.

FIG. 4 shows a diagnostic and treatment system according to a first embodiment. The system includes a treatment laser source device 10, a detection device 50 and a feedback device 80. The laser source device 10 emits a treatment laser beam 1 towards the skin 8 to be treated. In an exemplary embodiment, the laser beam 1 is temporally continuous. Preferably, the laser beam 1 is a pulse beam with pulses of duration comprised between 30 ps and 10 ns. The laser pulse rate may vary between 500 MHz and 500 kHz. This temporal operating domain allows using a laser pulse having both a high peak power and a relatively high mean power. This laser pulse allows combining photo-ablative effects coupled to thermal effects in a particularly innovating manner in dermatology. This selection of a duration and rate temporal domain for the laser pulses opens the way to a new kind of interaction in which the photo-ablative effects and the thermal effects may play complementary roles. This source is based on a so-called mode-locked, high rate (between 10 MHz and 500 MHz) laser architecture. The mode-locked regime may be obtained by the use of a saturable absorber, a modulator or a non-linear rotation of the polarization. The association of this source with an element allowing the extraction of one or several pulses (acousto-optical or electro-optical modulator, Pockels cell) generates a single pulse or packets of several variable-rate pulses. The duration of the pulse is adjusted by modifying the dispersion of the signal emitted by means of a dispersive element (volume or fibre Bragg gratings, dispersive mirror, prism).

In the spectral range, the laser source device 1 used for the treatment generates several discrete wavelengths or is continuously tunable over a wideband in the visible and/or infrared domains. This is made by inserting in the laser cavity a filter for the infrared radiation. The visible radiation is generated by frequency doubling.

A feedback device 80 is arranged between the photo-detection device 50 and the treatment laser source device 10. A computing processor analyses in real time the absorption measurements of the tissue 8 in order to determine the wavelength adapted to the skin or to the tattoo. The analysed information is then transmitted to the driver software of the laser source device 1 that is adapted to the wavelength as a function of the absorption peak of the chromophore, the tissue or the tattoo to be treated. As an alternative, the selection of the treatment wavelength is performed before the treatment. For that purpose, a spectral image of the area to be treated is for example acquired, with a recording of the different wavelengths to be provided for the treatment laser as a function of each point of the image.

Method

In FIG. 5 is shown an example of dermatological laser treatment system and method. In a first step, another light source 9 generates a low-intensity light beam 19 towards the skin 8 to be analysed. The photo-detection 5 and spectrometry 6 system measures a reflection spectrum of the skin 8. A computer or a microcontroller 70 selects a wavelength, a duration (comprised between 30 ps and 10 ns) and a repetition rate (between 10 MHz and 500 MHz) of the treatment laser 10 as a function of the absorption spectrum measured. A train of treatment laser pulses is applied to the skin 8.

The other illumination source 9 of the skin sample may be a coherent or non-coherent source emitting a wide spectrum or at least one discrete wavelength. This other light source 9 may be the treatment laser 10 but configured to deliver a low-power and/or low-energy light beam 19 in order not to degrade or deteriorate the skin sample. The light beam 19 illuminates the skin 8 to be analysed and treated. The reflected and/or backscattered beam is collected by means of the collecting device and measured by means of the spectrometry 6 and photo-detection 5 device comprising one or several photo-diodes. The variations of the absorption measurements (deduced from the reflection and/or backscattering measurements) are recorded. It is deduced therefrom one or several spectral bands of absorption mainly by the skin 8. These spectral bands of absorption correspond to the different tattoo colours, to the melanin of the skin and/or to markers used in photodynamic therapy (PDT). The optimum conditions of laser treatment are deduced from this analysis.

A feedback loop 80 is then coupled with the treatment laser source device 10. The treatment laser source device 10 comprises a laser tunable in wavelength in the visible and infrared spectral domains. As an alternative, the treatment laser source device 10 includes several single-wavelength lasers in the visible and infrared spectral domains. These lasers are continuous or pulse lasers as a function of the applications. Preferably, the treatment laser source device 10 emits laser pulses of duration comprised between 500 femtosecond and 100 microseconds with rates from 1 Hz to 1 GHz. The treatment laser beam has a mean power comprised between 100 mW and 100 W.

The laser treatment system controlled in real time allows performing many dermatological treatments, such as tattoo removal, depilation, photo-rejuvenation or photodynamic therapy.

This laser treatment system also allows determining in real time the treatment progress thanks to the amplitude of the collected signal linked to the absorption of the surface. For example, in a detattooing process, the more reduced the pigment concentration, the lower the amplitude of the collected absorption signal. It is hence possible to stop the laser treatment thanks to a direct measurement in situ and not based on the simulations predicting the effects of the treatment.

In practice, the absorption spectrum of the skin may significantly vary from one patient to another according to his/her phototype. Moreover, from one skin to another, the thermal diffusivity and thermal conductivity coefficients also vary. Hence, the thermal effects induced by a same treatment may vary from one patient to another.

To control the thermal effects, FIG. 6 illustrates a variant of a laser treatment system. This system includes a pulse laser source tunable in duration and repetition frequency to perform a thermally controlled laser treatment.

This type of temporal operation has a particularly innovative interest in dermatology, where the combination between relatively high peak power AND mean power in the pulse opens up the way to a new kind of interaction in which the photo-ablative effects coupled to thermal effects may play complementary roles.

The system of FIG. 6 includes a laser source 10 tunable in repetition rate. The laser source 10 generates laser pulses or pulse packets. This system is based on a so-called mode-locked laser architecture. The mode-locked regime may be obtained by the use of a saturable absorber, a modulator or a non-linear rotation of the polarization. The association of this source with an element allowing the extraction of one or several pulses (acousto-optical or electro-optical modulator, Pockels cell) generates a single pulse or packets of several pulses.

The laser treatment system herein includes a thermal sensor 90 incorporated in the laser head in contact with the epidermis. The thermal sensor 90 measures the temperature on the treated skin. This information allows adapting the thermal load applied to the patient during the laser treatment in order to avoid the damages generated by a too high temperature and/or duration of exposure.

Indeed, FIG. 7 shows a curve (a) representative of the appearance of deep burns, respectively the curve (b) of superficial burns, and the curve (c) of a discomfort, as a function of the duration of exposure to the laser treatment and of the surface temperature of the skin.

1. A dermatological diagnostic and treatment system comprising:
a light source device (2),
a light flow collecting device (4),
an absorption spectroscopic measurement device (5, 6), and
a calculator,
wherein:
the light source device (2) is adapted to generate a first low-intensity light beam (20) towards an epidermal surface (8) of a patient, the first light beam (20) extending over a spectral range or being tunable in wavelength over a spectral range,
the light flow collecting device (4) being arranged to collect a first light flow by reflection and/or diffusion of the first light beam (20) on said epidermal surface (8),
the absorption spectroscopic measurement device (5, 6) is adapted to record a first spectroscopic measurement of the first light flow as a function of the spectral range, and
the calculator being adapted to receive the first spectroscopic measurement in the spectral range and to determine a first wavelength associated with an absorption of a pigment of said epidermal surface and a second wavelength associated with an absorption of another tissue constituent of said epidermal surface, and in that
the light source device includes a laser source (10) tunable in wavelength inside the spectral range, the laser source being tunable in duration and/or rate and the laser source being adapted to generate a second treatment laser beam (1) of high intensity at said first wavelength towards said epidermal surface (8) and in that the dermatological diagnostic and treatment system further includes a temperature sensor and a feedback device (80, 90, 95), the temperature sensor being arranged to record a temperature measurement signal of said epidermal surface as a function of the application of the second laser beam and the feedback device (80, 90, 95) being configured to modify the duration and/or rate of the laser pulses as a function of the temperature measurement signal.

2. The dermatological diagnostic and treatment system according to claim 1, wherein the second laser beam (1) is consisted of pulses of duration comprised between 30 ps and 10 ns.

3. The dermatological diagnostic and treatment system according to claim 1, wherein the light flow collecting device (4) includes an integrating sphere, and wherein the absorption spectroscopic measurement device (5, 6) includes a photo-detection device (5) and a spectrometer or a monochromator.

4. The dermatological diagnostic and treatment system according to claim 1, wherein the laser source (10) is continuously tunable in wavelength over a spectral range extending over at least 100 nm.

5. The dermatological diagnostic and treatment system according to claim 4, wherein the laser source (10) is adapted to generate the first light beam (20) and the second laser beam (1) and wherein the light source device (2) further includes a device for intensity modulating the laser beam generated by the laser source.

6. The dermatological diagnostic and treatment system according to claim 1, wherein the light source device (2) includes another light source adapted to generate the first light beam (20).

7. The dermatological diagnostic and treatment system according to claim 1, wherein the light flow collecting device (4) is configured to collect a second light flow (50) by reflection and/or scattering of the second light beam (1) on said epidermal surface (8) and wherein the absorption spectroscopic measurement device (5, 6) is adapted to record a second light flow measurement signal at the first wavelength.

8. The dermatological diagnostic and treatment system according to claim 7, wherein the feedback device (80, 90, 95) is configured to modify the duration and/or the rate of the laser pulses as a function of the second light flow measurement signal.

9. The dermatological diagnostic and treatment system according to claim 1, wherein the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

10. The dermatological diagnostic and treatment system according to claim 3, wherein the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

11. The dermatological diagnostic and treatment system according to claim 4, wherein the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

12. The dermatological diagnostic and treatment system according to claim 5, wherein the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

13. The dermatological diagnostic and treatment system according to claim 6, wherein the spectroscopic measurement device includes an image detector having a pixel array adapted to form an hyperspectral image of said epidermal surface in said spectral range, and the calculator being adapted to determine the second laser treatment wavelength corresponding to each pixel of the image detector from said hyperspectral image.

14. The dermatological diagnostic and treatment system according to claim 4, wherein the light source device (2) includes another light source adapted to generate the first light beam (20).
